(19) United States
(12) Patent Application Publication
(10) Pub. No.: US 2015/0080776 A1
(43) Pub. Date: Mar. 19, 2015

(54) SLING SYSTEM FOR PROVIDING BOTH RELIABLE IMMOBILIZATION AND COMFORT

(71) Applicant: Lisa Torché Davis, Chittenango, NY (US)

(72) Inventor: Lisa Torché Davis, Chittenango, NY (US)

(21) Appl. No.: 14/028,584

(22) Filed: Sep. 17, 2013

(51) Int. Cl. A61F 5/37

(52) U.S. Cl. CPC A61F 5/3738
USPC 602/4

(57) ABSTRACT

A sling system for providing both reliable immobilization and comfort is disclosed. The sling system comprises a lightweight elastic sling, a support wedge, and a restraint band for securing the user's hand and/or arm to the support wedge, while at the same time the user's shoulder and arm are supported in the sling. The sling comprises various pleats and darts, such as the shoulder compression pleats, that allow the sling to be form-fitted snuggly and comfortably in cocoon-like fashion around the shoulder, arm, and elbow of the user, thereby securely and comfortably immobilizing the injured or repaired shoulder and/or arm and redistributing the weight of the arm away from the shoulder alone, which may be injured, to the combination of both the shoulder and the torso.

### TECHNICAL FIELD

The presently disclosed subject matter relates generally to medical support slings and more particularly to a sling system for providing both reliable immobilization and comfort.

### BACKGROUND

Various shoulder and arm supports exist today that are used in the medical field for shoulder and arm treatment. For example, arm slings are used by post trauma patients to treat the shoulder and/or arm. In one example, following rotator cuff surgery, the recovering patient is required to wear an arm sling to relieve the weight of the patient's arm from the surgically repaired shoulder. The arm sling must be worn continuously 24 hours per day for several weeks, requiring the recovering patient to wear the arm sling even when sleeping.

Certain drawbacks exist with respect to conventional arm slings for shoulder and arm support. Examples of the drawbacks of conventional arm slings include, but are not limited to, (1) they can be uncomfortable when wearing for extended periods of time, (2) they can be particularly uncomfortable when wearing while sleeping, (3) they may not be reliably secure when worn while sleeping (i.e., they tend to loosen and slip off during sleep), (4) they can restrict the use of the hand or fingers of the supported arm, (5) when supported by the opposite shoulder, they can restrict the use of the non-injured arm or hand, (6) they may not adequately ensure the immobilization of the supported arm or shoulder when accidentally bumped or jarred, (7) if not custom fitted, they may not provide ideal support, and (8) they can be aesthetically unpleasing to the eye.

Because of the aforementioned drawbacks to conventional shoulder and arm supports, (1) the shoulder and arm supports can have limited effectiveness, and (2) recovering patients tend not to wear their shoulder and arm supports as prescribed. As a result, the efficacy of the shoulder and arm supports with respect to assisting the healing process can be compromised and, further, there is risk of reinjuring the shoulder or arm.

### SUMMARY

In one aspect, the presently disclosed subject matter provides a sling system with reliable immobilization and comfort for supporting an injured arm or shoulder comprising: a) a generally rectangular or oval pouch for receiving a subject's arm and shoulder comprising a shoulder surround portion situated lengthwise along the top of the pouch and an arm surround portion situated lengthwise along the bottom of the pouch and having a narrowed first end and a second end each comprising a fastener; wherein the first and second ends fasten around the torso of the subject at each fastener; and wherein the elbow of the subject is completely covered by the pouch; b) the arm surround portion further comprising an arm flap comprising an arm flap fastener to stretch around the arm on the injured side of the subject; c) the shoulder surround portion further comprising at least one shoulder compression pleat; d) a support wedge having a generally elongated block shape and comprising a narrow end that is oriented towards the back of the subject's torso and a wide end oriented towards the front of the subject's torso; and e) a restraint band for securing the subject's hand on the injured side of the subject to the support wedge.

In another aspect, the presently disclosed subject matter provides a method for a subject to secure a sling on himself/herself, the method comprising: a) providing a sling system comprising: i) a pouch for receiving a subject's arm and shoulder comprising a shoulder surround portion and an arm surround portion and having a first end and a second end each comprising a fastener; wherein the first and second ends fasten around the torso of the subject at each fastener; and wherein the elbow of the subject is completely covered by the pouch; ii) the arm surround portion further comprising an arm flap comprising an arm flap fastener to stretch around the arm on the injured side of the subject; iii) the shoulder surround portion further comprising at least one shoulder compression pleat; iv) a support wedge comprising a narrow end that is oriented towards the back of the subject's torso and a wide end oriented towards the front of the subject's torso; and v) a restraint band for securing the subject's hand on the injured side of the subject to the support wedge; b) placing the pouch onto a surface with the inside of the pouch facing upward; c) picking up the pouch with the support wedge inside; d) holding the pouch and the support wedge in the approximate desired position to allow securing of the support wedge around the torso of the subject; e) securing the support wedge into place around the torso of the subject; f) positioning the at least one shoulder compression pleat in the approximate desired position with respect to the shoulder; g) reaching around the back of the subject to grasp one end of the pouch and pulling that end of the pouch around the torso toward the other end of the pouch; h) bringing the first end and the second end of the sling together and fastening them loosely together; i) adjusting the pouch and the support wedge into position; and j) securing the arm flap around the arm on the injured side of the subject.

In still another aspect, the presently disclosed subject matter provides a method for a subject to secure a sling on himself/herself, the method comprising: a) providing a sling system comprising: i) a pouch for receiving a subject's arm and shoulder comprising a shoulder surround portion and an arm surround portion and having a first end and a second end each comprising a fastener; wherein the first and second ends fasten around the torso of the subject at each fastener; and wherein the elbow of the subject is completely covered by the pouch; ii) the arm surround portion further comprising an arm flap comprising an arm flap fastener to stretch around the arm on the injured side of the subject; and iii) the shoulder surround portion further comprising at least one shoulder compression pleat; b) picking up the pouch and positioning the at least one shoulder compression pleat in the approximate desired position with respect to the shoulder; c) reaching around the back of the subject to grasp one end of the pouch and pulling that end of the pouch around the torso toward the other end of the pouch; d) bringing the first end and the second end of the sling together and fastening them loosely together; e) adjusting the pouch into position; and f) securing the arm flap around the arm on the injured side of the subject.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 illustrates a perspective view of a subject wearing the presently disclosed sling system for providing both reliable immobilization and comfort;

FIG. 2 illustrates a perspective view of an embodiment of the sling of the presently disclosed sling system;

FIG. 3 illustrates another perspective view of an embodiment of the sling of the presently disclosed sling system;

FIG. 4 illustrates a perspective view of a subject wearing an embodiment of the support wedge of the presently disclosed sling system;

FIG. 5 illustrates a perspective view of a subject wearing both the support wedge portion and the restraint band portion of the presently disclosed sling system;

FIG. 6 illustrates a perspective view of a subject wearing the sling only of the presently disclosed sling system;

FIG. 7 illustrates another perspective view of a subject wearing the sling only of the presently disclosed sling system;

FIG. 8 illustrates a perspective view of the back of a subject wearing the sling only of the presently disclosed sling system;

FIG. 9 illustrates another perspective view of a subject wearing the sling only of the presently disclosed sling system;

FIG. 10 illustrates a plan view of an embodiment of a pattern for making the sling of the presently disclosed sling system;

FIG. 11 illustrates a plan view of another embodiment of a pattern for making the sling of the presently disclosed sling system;

FIG. 12 illustrates a plan view of still another embodiment of a pattern for making the sling of the presently disclosed sling system;

FIG. 13 illustrates a flow diagram of an embodiment of a method of putting on the presently disclosed sling system; and

FIG. 14 illustrates a flow diagram of another embodiment of a method of putting on the presently disclosed sling system.

### DETAILED DESCRIPTION

The presently disclosed subject matter now will be described more fully hereinafter with reference to the accompanying Drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a sling system for providing both reliable immobilization and comfort. Namely, the sling system is a system for supporting the shoulder and arm of a subject who is wearing the sling system. The sling system provides reliable immobilization (even when sleeping) of the shoulder and arm of the subject who is wearing the sling system. Additionally, the sling system is comfortable to wear. The sling system comprises a lightweight elastic sling, a support wedge, and a restraint band for securing the subject's hand and/or arm to the support wedge, while at the same time the sling is supporting the subject's shoulder and arm. Because the sling system provides both reliable immobilization and comfort, patient compliance with respect to wearing shoulder and arm supports as prescribed can be significantly improved as compared with conventional shoulder and arm supports (e.g., conventional slings).

The sling, the support wedge, and the restraint band of the presently disclosed sling system can be worn in various combinations depending on the particular needs of the subject. In some embodiments, the subject wears the sling, the support wedge, and the restraint band. In other embodiments, the subject wears the sling and the support wedge, but does not wear the restraint band. In yet other embodiments, the subject wears the sling only, without the support wedge and without the restraint band.

An aspect of the presently disclosed sling system is that it is worn on and supported by the injured shoulder or the shoulder of the injured arm; namely, the sling system is not worn on and supported by the shoulder that is opposite the injured shoulder and/or arm, thereby posing no restrictions to the healthy, uninjured shoulder and arm.

Another aspect of the presently disclosed sling system is that the sling thereof wraps around the entire shoulder and arm as well as around the torso. In so doing, the weight of the arm is redistributed away from the shoulder alone and to the combination of both the shoulder and the torso, thereby relieving the injured shoulder of much of the weight of the arm. More specifically, while the shoulder is gently compressed within the confines of the sling, the arm is being lifted and carried within the sling, wherein the sling is wrapped around the torso in such a way that the majority of the weight of the arm is redistributed away from the shoulder to the torso.

Yet another aspect of the presently disclosed sling system is that the sling thereof wraps around the entire shoulder and arm, holding the shoulder and arm in the prescribed position, wherein the sling system is easily adjustable to achieve the uniquely prescribed tilt or angle that each subject may require for healing.

Yet another aspect of the presently disclosed sling system is that the sling thereof includes full shoulder compression pleats that provide a comforting, gentle, and even pressure directly on the injured or repaired shoulder, thereby providing incentive for subjects to wear the sling as prescribed that in turn ensures efficacy of the sling system with respect to immobilizing and healing the injured or repaired shoulder and/or arm.

Yet another aspect of the presently disclosed sling system is that the sling thereof provides cocoon-like protection of the elbow, thereby reducing, preferably entirely eliminating, the likelihood of the subject's elbow accidentally catching on obstacles, which in turn ensures efficacy of the sling system with respect to immobilizing and healing the injured or repaired shoulder and/or arm when accidentally bumped or jarred.

Yet another aspect of the presently disclosed sling system is that, by strapping the arm/hand to the support wedge, the wearer's natural reflex to reach for things is mitigated, thereby ensuring efficacy of the sling system with respect to immobilizing and healing the injured or repaired shoulder and/or arm.

Yet another aspect of the presently disclosed sling system is that it is comfortable even when sleeping because it is lightweight and does not include bulky straps and bulky fasteners, thereby providing incentive for subjects to wear the sling as prescribed that in turn ensures efficacy of the sling system with respect to immobilizing and healing the injured or repaired shoulder and/or arm.

Yet another aspect of the presently disclosed sling system is that, by strapping the arm/hand to the support wedge, the wearer's injured or repaired shoulder and/or arm is held reliably immobilized even when sleeping. Namely, the wearer's injured or repaired shoulder and/or arm cannot accidently slip out of the presently disclosed sling system during sleep.

Yet another aspect of the presently disclosed sling system is that the sling thereof can be made aesthetically pleasing to the eye, thereby providing incentive for subjects to wear the sling as prescribed that in turn ensures efficacy of the sling system with respect to immobilizing and healing the injured or repaired shoulder and/or arm.

Yet another aspect of the presently disclosed sling system is that the sling thereof allows the hand of the injured resting arm to be used for non-lifting, non-pressure types of tasks, such as holding small objects.

Still another aspect of the presently disclosed sling system is that the sling thereof may include other features, such as pockets for holding small items and/or pockets for holding ice packs.

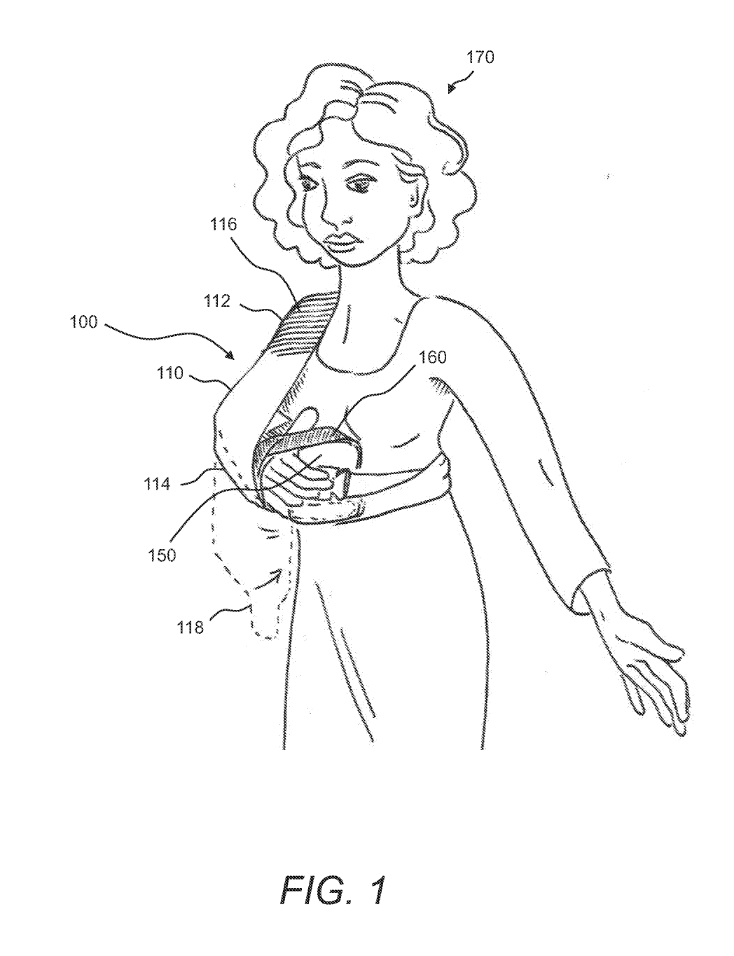

Referring now to FIG. 1, in some embodiments, the presently disclosed subject matter provides a sling system 100 for providing reliable immobilization of the subject's shoulder and arm as well as being comfortable to wear. FIG. 1 shows a subject 170 wearing the presently disclosed sling system 100, whereas the subject 170 is any individual who is the subject of treatment for an injured or repaired shoulder and/or arm.

The sling system 100 comprises a sling 110, a support wedge 150, and a restraint band 160 for securing the hand and/or arm of subject 170 to the support wedge 150, while at the same time the shoulder and arm of subject 170 are supported in the sling 110. The sling system 100 is designed to provide reliable immobilization of the shoulder and arm of the subject 170. At the same time, the sling system 100 is designed to be highly comfortable as compared with conventional shoulder and arm supports. Therefore, the sling system 100 provides incentive to wear the sling system 100 as prescribed, which in turn ensures efficacy of the sling system 100 with respect to immobilizing the injured or repaired shoulder and/or arm.

The sling 110, the support wedge 150, and the restraint band 160 of the presently disclosed sling system 100 can be worn in various combinations depending on the particular needs of the subject 170. In some embodiments, the subject 170 wears the sling 110, the support wedge 150, and the restraint band 160. In other embodiments, the subject 170 wears the sling 110 and the support wedge 150, but does not wear the restraint band 160. In yet other embodiments, the subject 170 wears the sling 110 only, without the support wedge 150 and without the restraint band 160. While the sling system 100 is shown in FIG. 1 in relation to the right shoulder/arm, it is understood that the sling system 100 can be designed for use with the left shoulder/arm.

The sling 110 includes a shoulder surround portion 112 and an arm surround portion 114. The shoulder surround portion 112 further includes a set of shoulder compression pleats 116. In some embodiments, the shoulder compression pleats 116 are full shoulder compression pleats that provide a comforting, gentle, and even pressure directly on the injured or repaired shoulder, thereby providing incentive for subjects to wear the sling system 100 as prescribed, which in turn ensures efficacy of the sling system 100 with respect to immobilizing the injured or repaired shoulder and/or arm.

The arm surround portion 114 further includes an arm flap 118. In some embodiments, when the support wedge 150 is used, the arm flap 118 stretches around both the support wedge 150 and the arm of subject 170. In other embodiments, when the support wedge 150 is used, the arm flap 118 stretches around only the arm of subject 170 and then the arm rests upon or against the support wedge 150. In yet other embodiments, when the support wedge 150 is not used, the arm flap 118 stretches around only the arm of subject 170.

The sling 110 is formed of any lightweight elastic material or fabric that allows the sling 110 to be easily stretched around the torso of the subject 170. In some embodiments, the sling 110 is formed of spandex based fabrics, including but not limited to, cotton spandex, polyester spandex, acetate spandex, and any combinations thereof.

Optionally, the sling 110 can include a lining. In other embodiments, the lining is formed of cotton spandex, polyester spandex, acetate spandex, and the like. In still other embodiments, the sling 110 is formed of fabric-sided thin neoprene.

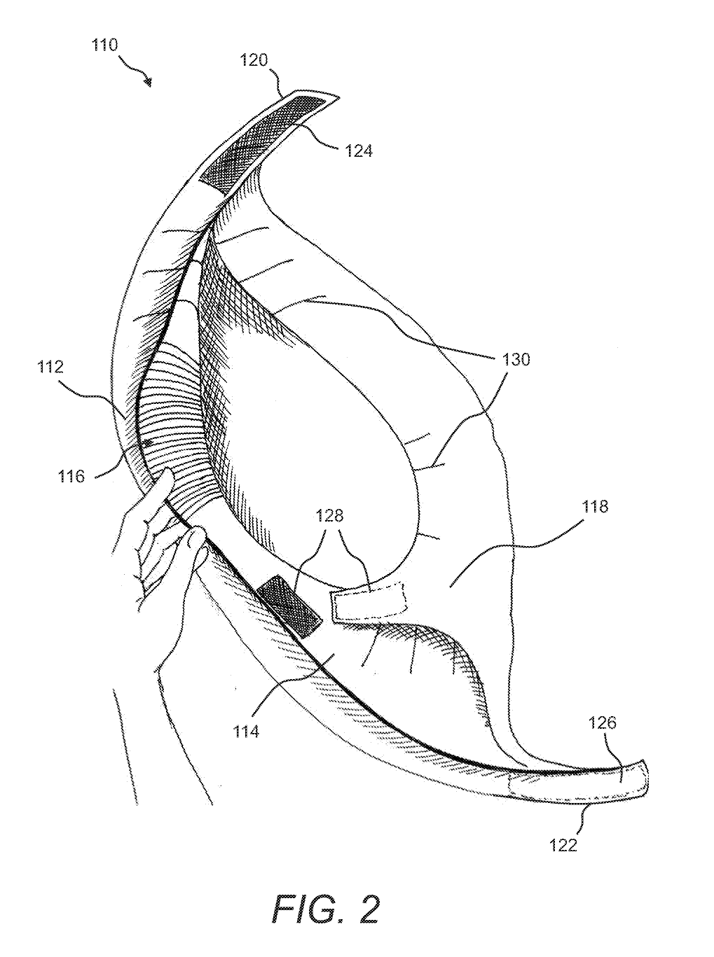

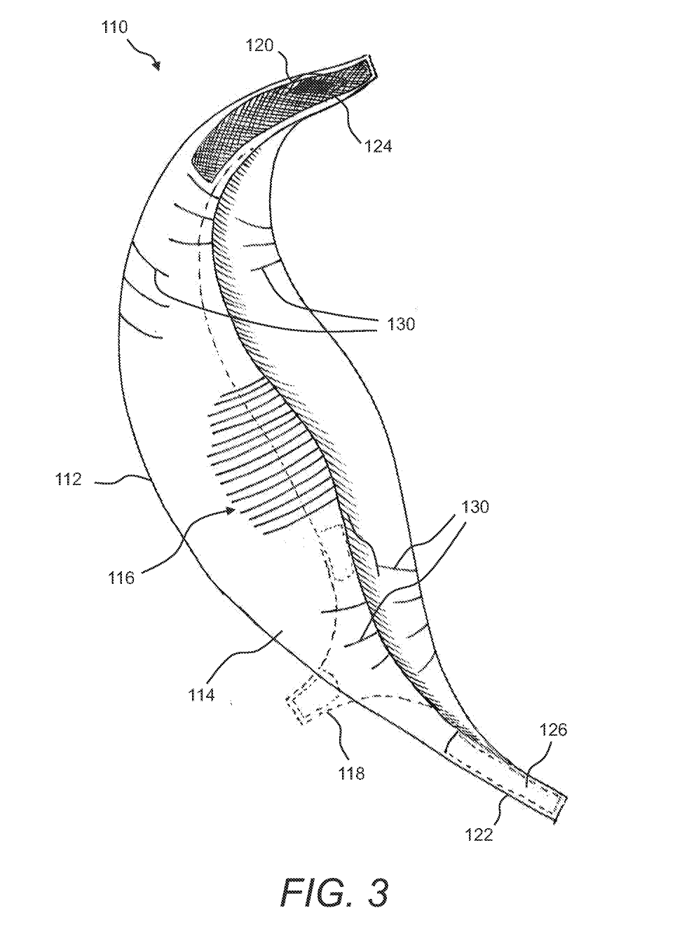

Additionally, the sling 110 can be provided in various sizes depending on the size of the subject 170. Namely, because the sling 110 is formed of a stretchable material or fabric, the sling 110 can be provided in, for example, small, medium, large, and extra-large sizes, yet still conform to the body of each subject 170 and fit properly. That is, the sling system 100 does not require a custom-fitted sling 110 to ensure efficacy with respect to immobilizing the injured or repaired shoulder and/or arm. More details of the sling 110 are shown and described with reference to FIG. 2 and FIG. 3.

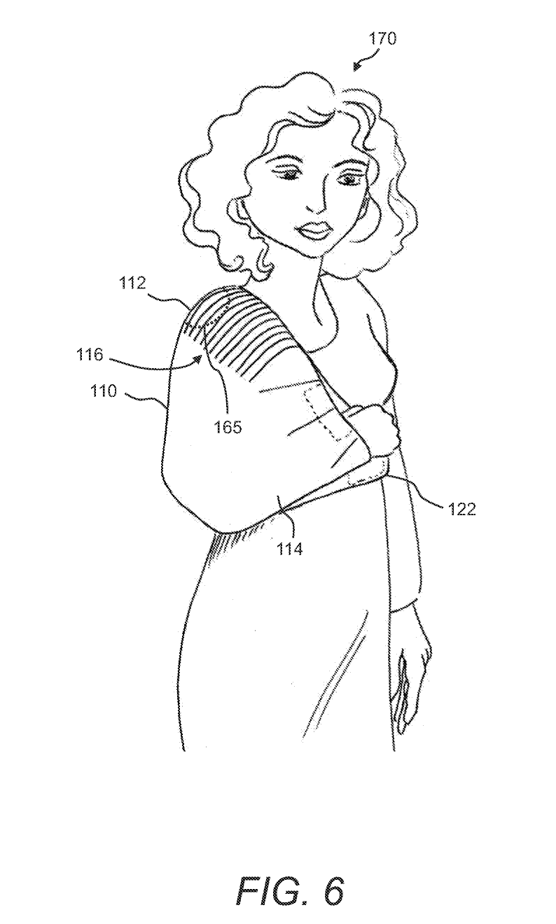

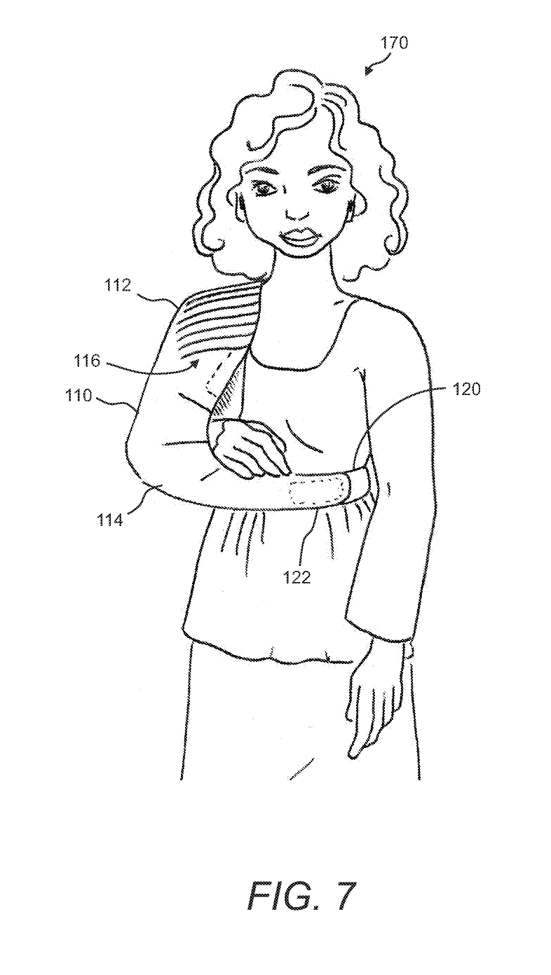

FIG. 2 and FIG. 3 show perspective views of an embodiment of the sling 110 of the presently disclosed sling system 100. The sling 110 has a first end 120 and a second end 122. When the sling 110 is stretched around the torso of the subject 170, the first end 120 and second end 122 can be fastened together to hold the sling 110 in place, as shown in FIGS. 6 to 9. In some embodiments, the sling 110 includes a fastener 124 at the first end 120 and a fastener 126 at the second end 122, wherein the fastener 124 at the first end 120 can engage with the fastener 126 at the second end 122. In one example, the fastener 124 and the fastener 126 are formed of Velcro®, as shown. However, the fasteners 124 and 126 are not limited to Velcro®. The fasteners 124 and 126 can be any fastening mechanisms, such as, but not limited to, buttons, snaps, hooks, ties, and the like.

FIG. 2 and FIG. 3 also show that the sling 110 includes an arm flap fastener 128. In some embodiments, the arm flap fastener 128 is formed by a piece of Velcro® on the end of the arm flap 118 and another piece of Velcro® on the inside of the arm surround portion 114 of the sling 110. However, the arm flap fastener 128 is not limited to Velcro®. The arm flap fastener 128 can be any fastening mechanism, such as, but not limited to, buttons, snaps, hooks, ties, and the like.

In addition to the shoulder compression pleats 116, the body of the sling 110 also includes various other pleats/darts 130. Pleats and darts, such as the shoulder compression pleats 116 and the other pleats/darts 130, allow the sling 110 to be form-fitted snuggly and comfortably in cocoon-like fashion around the shoulder, arm, and elbow of the subject 170, thereby securely and comfortably immobilizing the injured or repaired shoulder and/or arm.

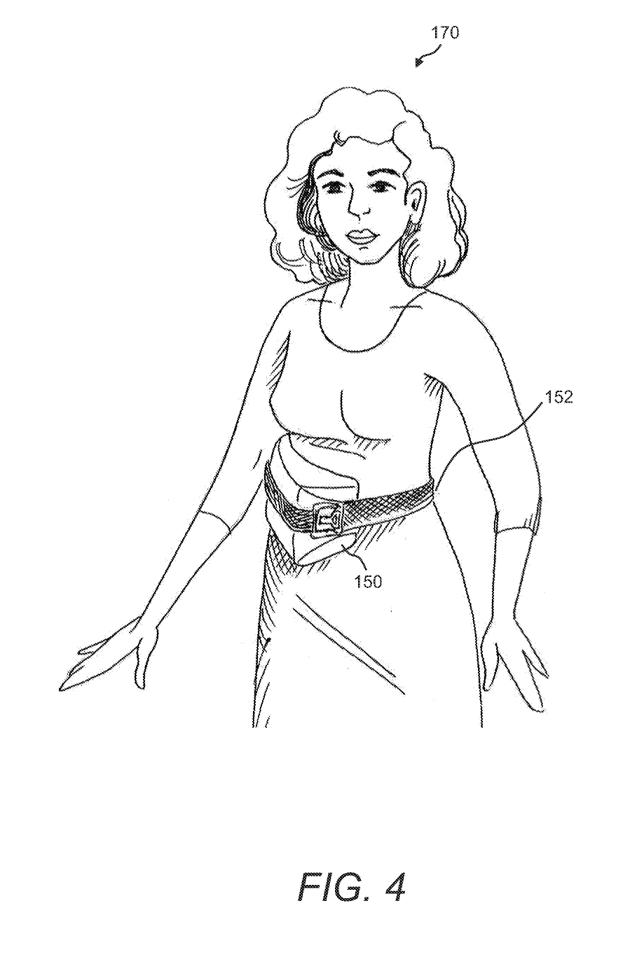

FIG. 4 is a perspective view of a subject 170 wearing an embodiment of the support wedge 150 of the presently disclosed sling system 100. The support wedge 150 is used to hold the injured or repaired shoulder and/or arm at the proper angle for healing. In some embodiments, the support wedge 150 is used to hold the humerus in the neutral anatomic position. By “neutral anatomic position”, it is meant that the humerus is in the approximate position it would be if the arm was hanging by the subject's side. The subject 170 wears the support wedge 150 such that the narrow end of the support wedge 150 is oriented toward the back of his/her torso and the wide end of the support wedge 150 is oriented toward the front of his/her torso. The support wedge 150 can be secured around the torso of the subject 170 using, for example, an adjustable support wedge strap 152.

The support wedge 150 can be, for example, any standard support wedge that is used with other shoulder and arm supports. In some embodiments, the support wedge 150 is the Breg® Neutral Wedge supplied by Breg, Inc. (Carlsbad, Calif.).

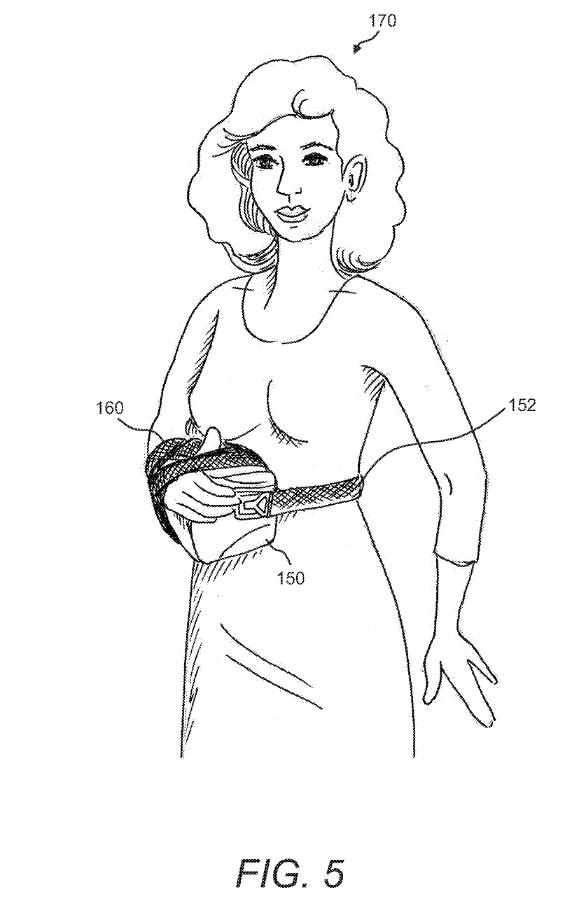

FIG. 5 is a perspective view of a subject 170 using the restraint band 160 to secure his/her hand to the support wedge 150. The restraint band 160 can be any elastic band suitable for holding the arm/hand of the subject 170 securely to the support wedge 150. In some embodiments, the width of the restraint band 160 is from about 1 inch to about 2 inches. In other embodiments, the length of the restraint band 160 is from about 4 feet to about 6 feet. In still other embodiments, the restraint band 160 is an ACE™ Brand Bandage. Subsequent to securing the arm/hand to the support wedge 150 using the restraint band 160, the sling 110 can be put on and the arm flap 118 wrapped around both the support wedge 150 and the arm of the subject 170, as shown in FIG. 1.

FIGS. 6 to 9 show various perspective views of a subject 170 wearing the sling 110 of the presently disclosed sling system 100 without the support wedge 150 and without the restraint band 160. Namely, FIGS. 6 to 9 show the sling 110 stretched around the torso of the subject 170 and with the first end 120 and second end 122 fastened together to hold the sling 110 in place.

By way of example, FIGS. 6 to 9 show the sling 110 being worn on the right side of the body of subject 170, wherein the right shoulder and/or arm is the injured or repaired shoulder and/or arm. In some embodiments, the sling 110 is worn on and supported by the injured or repaired shoulder and arm, which in this example is the right shoulder and arm. As a result, the sling 110 poses no restrictions to the healthy left shoulder and arm.

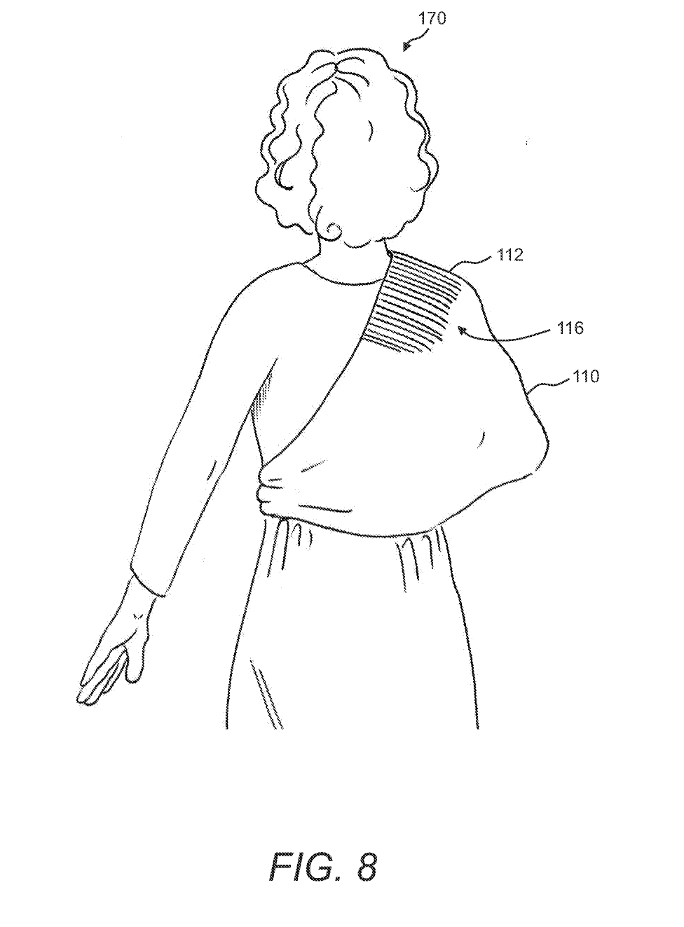

Further, because the sling 110 wraps around the entire shoulder and arm as well as around the torso of the subject 170, the weight of the arm is redistributed away from the shoulder alone and to the combination of both the shoulder and the torso, thereby relieving the injured shoulder of much of the weight of the arm. Namely, while the shoulder is gently compressed within the confines of the sling 110, the arm is being lifted and carried within the sling 110, wherein the sling 110 is wrapped around the torso in such a way that the majority of the weight of the arm is redistributed away from the shoulder to the torso.

Whereas the elbow can be exposed using conventional shoulder and arm supports, FIG. 8 shows that the sling 110 provides cocoon-like protection of the elbow, thereby reducing, preferably entirely eliminating, the likelihood of the elbow of the subject 170 accidentally catching on obstacles as compared with conventional shoulder and arm supports. This cocoon-like protection of the elbow ensures efficacy of the sling system 100 with respect to holding the injured or repaired shoulder and/or arm immobile when accidentally bumped or jarred. For example, FIG. 8 shows a view of the sling 110 from the back of the subject 170. This view shows that the elbow of the subject 170 is completely enclosed and protected within the elastic fabric of the sling 110. By contrast, in conventional shoulder and arm supports (e.g., conventional slings), the weight of the elbow is supported; however, there is no protection against the subject accidently catching his/her elbow on an obstacle or against sudden jarring of the elbow if accidently bumped. In the sling system 100, the sling 110 provides protection against the subject accidently catching his/her elbow on an obstacle and against sudden jarring of the elbow if accidently bumped.

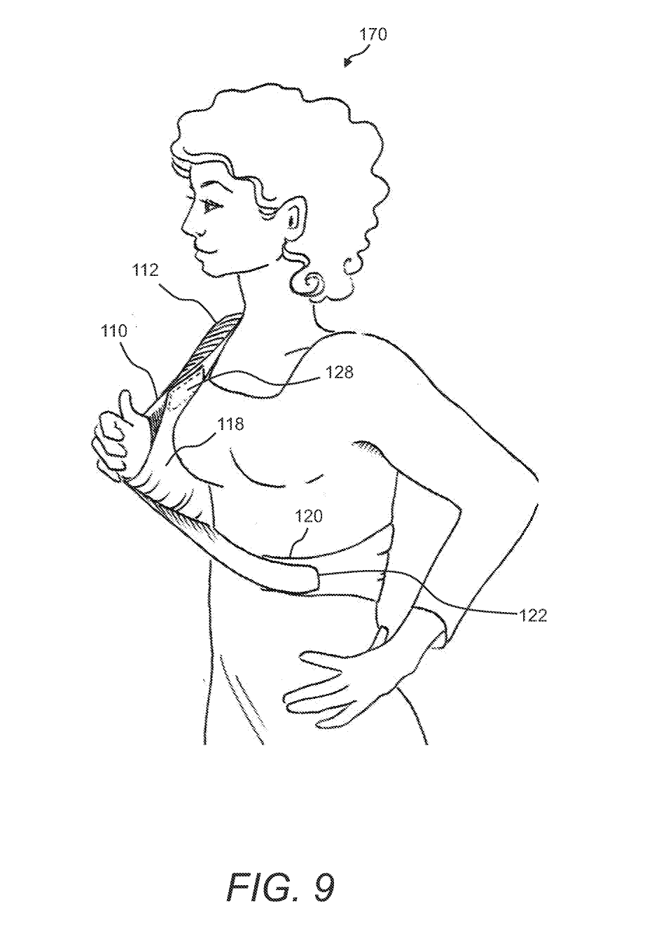

FIG. 9 shows a view of the arm flap 118 of the sling 110, wherein the arm flap 118 is wrapped around the arm of the subject 170 and secured via the arm flap fastener 128. While FIG. 9 shows the arm flap 118 in use without the support wedge 150, the arm flap 118 is designed to wrap in similar fashion around both the arm and the support wedge 150. Namely, the end of the arm flap 118 can be pulled between the support wedge 150 and the torso of the subject 170, then pulled around the support wedge 150 and the arm, and then secured to the inside of the sling 110 via the arm flap fastener 128 (see FIG. 1).

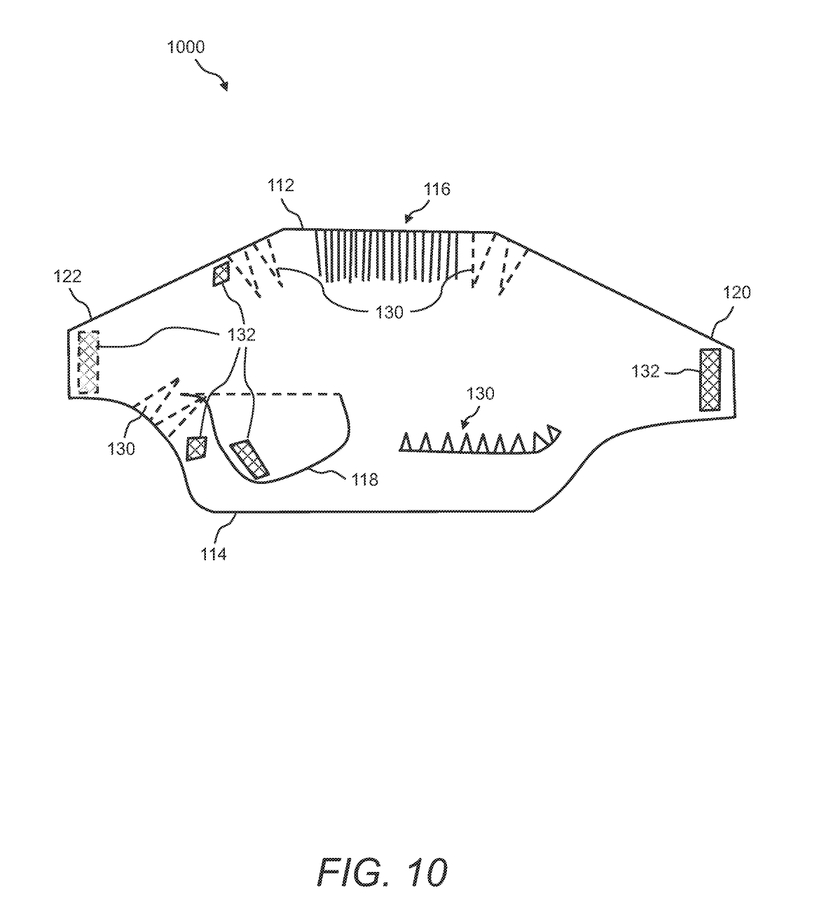

FIGS. 10 to 12 show plan views of embodiments of patterns for making the sling 110 of the presently disclosed sling system 100. One of the reasons that conventional shoulder and arm supports are not worn as prescribed is their poor visual appearance. In order to motivate subjects to wear the sling system 100 as prescribed and thereby ensure efficacy of the sling system 100 with respect to immobilizing the injured or repaired shoulder and/or arm, the sling 110 can be made aesthetically pleasing to the eye. In some embodiments, the sling 110 can be formed as a woman's dressy garment that has a pleasing appearance and that can be worn with a woman's outfit. In other embodiments, the sling 110 can be formed as a sporty garment that has a pleasing appearance that can be worn by men or women.

FIG. 10 shows a plan view of a sling pattern 1000, which is an embodiment of a pattern for forming the sling 110 as a woman's dressy garment, hereafter called the “dressy” sling 110. In some embodiments, the dressy sling 110 can include an outer shell formed of cotton spandex, polyester spandex, or acetate spandex and an inner lining also formed of cotton spandex, polyester spandex, or acetate spandex. The outer shell and inner lining can be the same or different colors and/or patterns of colors. In other embodiments, the outer shell and the inner lining of the dressy sling 110 can be any colors or patterns of colors that are aesthetically pleasing to the eye and that can complement a woman's outfit. In still other embodiments, the dressy sling 110 includes any decorative trim.

FIG. 10 indicates the portions of the sling pattern 1000 that correlate to the shoulder surround portion 112, the arm surround portion 114, the shoulder compression pleats 116, the arm flap 118, the first end 120, the second end 122, and to the various pleats/darts 130 of the dressy sling 110. Also shown in FIG. 10 are various pieces of Velcro® 132 that form, for example, the fastener 124 at the first end 120, the fastener 126 at the second end 122, and the arm flap fastener 128. The various pleats/darts 130 facilitate the cocoon-like fit and the aesthetically pleasing look of the dressy sling 110. In some embodiments, the overall length of the sling pattern 1000 is about 62 inches and the overall height of the sling pattern 1000 is about 28 inches. However, the length of the ends of the sling pattern 1000 can be extended if the dressy sling 110 is designed to be tied rather than fastened via Velcro® 132.

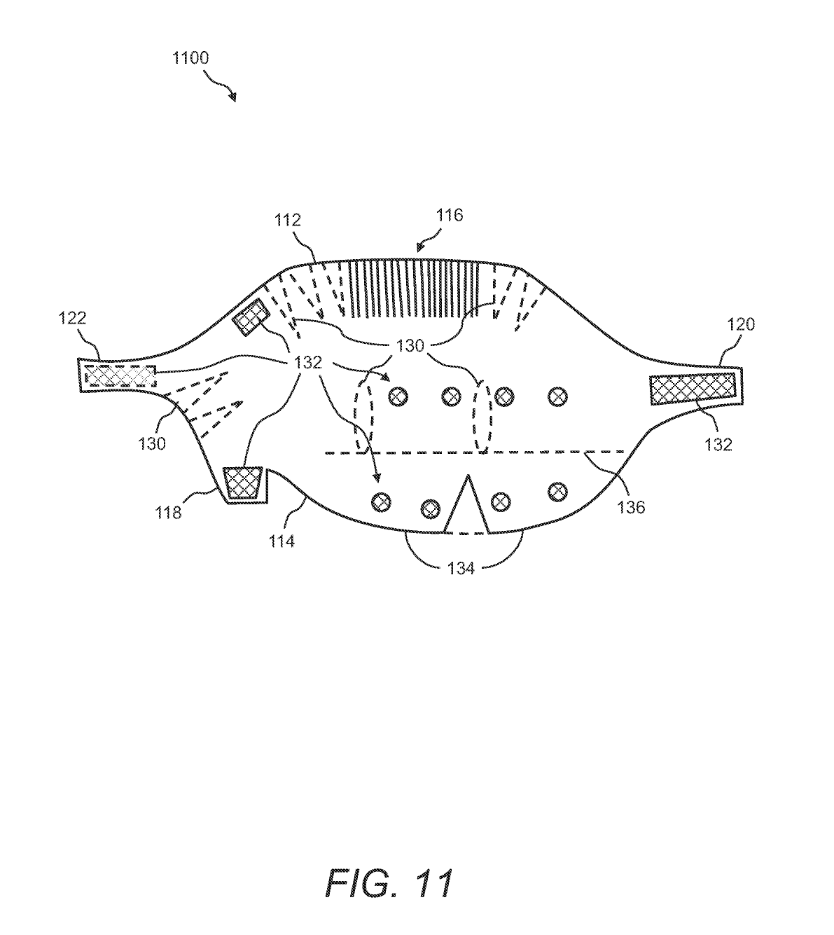

FIG. 11 shows a plan view of a sling pattern 1100, which is an embodiment of a pattern for forming the sling 110 as a sporty garment, hereafter called the “sporty” sling 110. In some embodiments, the sporty sling 110 includes a single piece of fabric-sided thin neoprene. In other embodiments, the fabric-sided thin neoprene is black.

FIG. 11 indicates the portions of the sling pattern 1100 that correlate to the shoulder surround portion 112, the arm surround portion 114, the shoulder compression pleats 116, the arm flap 118, the first end 120, the second end 122, and to the various pleats/darts 130 of the sporty sling 110. Also shown in FIG. 11 are various pieces of Velcro® 132 that form, for example, the fastener 124 at the first end 120, the fastener 126 at the second end 122, and the arm flap fastener 128. In some embodiments, the sporty sling 110 further includes a wedge flap 134. The wedge flap 134 is a flap that is separate from the arm flap 118. The wedge flap 134 is used to wrap around both the support wedge 150 and the arm of subject 170. In other embodiments, a fold 136 is provided to facilitate the function of the wedge flap 134. In still other embodiments, certain pieces of Velcro® 132 are provided (as shown) to hold the edge of the wedge flap 134 closed against the inside of the sporty sling 110. If the subject 170 is not using the support wedge 150 with the sporty sling 110, the wedge flap 134 is still folded up and held with the pieces of Velcro® 132. The various pleats/darts 130 facilitate the cocoon-like fit and the aesthetically pleasing look of the sporty sling 110. In further embodiments, the overall length of the sling pattern 1100 is about 58 inches and the overall height of the sling pattern 1100 is about 28 inches. In still further embodiment, when sewn, the sporty sling 110 is about 46 inches long and about 25 inches high.

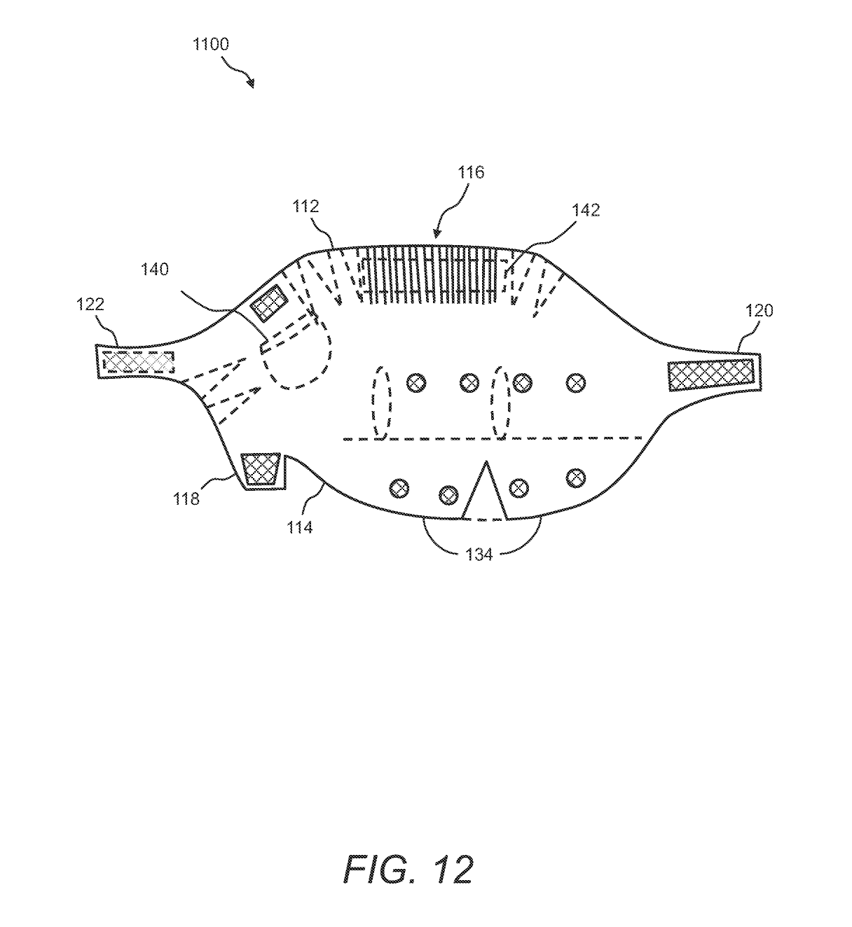

In some embodiments, the sling 110, such as the dressy sling 110 and the sporty sling 110, can include other features, such as, but not limited to, pockets for holding small items and pockets for holding ice packs. For example, FIG. 12 shows the sling pattern 1100 for forming the sporty sling 110, wherein the sporty sling 110 can further include a pocket 140 and an ice pocket 142. The pocket 140 can be used, for example, to hold a mobile phone, a small purse, and/or any small loose items. The ice pocket 142 can be used to hold an ice pack on the shoulder of the subject 170. For example, in the case of rotator cuff surgery, the location of the ice pocket 142 can coincide with the location of the surgical site. The pocket 140 and the ice pocket 142 can similarly be included in the dressy sling 110. Additionally, an ice pack can be held in the arm flap 118 of any type of sling 110.

Accordingly, in some embodiments, the presently disclosed subject matter provides a sling system with reliable immobilization and comfort for supporting an injured arm or shoulder comprising: a) a generally rectangular or oval pouch for receiving a subject's arm and shoulder comprising a shoulder surround portion situated lengthwise along the top of the pouch and an arm surround portion situated lengthwise along the bottom of the pouch and having a narrowed first end and a second end each comprising a fastener; wherein the first and second ends fasten around the torso of the subject at each fastener; and wherein the elbow of the subject is completely covered by the pouch; b) the arm surround portion further comprising an arm flap comprising an arm flap fastener to stretch around the arm on the injured side of the subject; c) the shoulder surround portion further comprising at least one shoulder compression pleat; d) a support wedge having a generally elongated block shape and comprising a narrow end that is oriented towards the back of the subject's torso and a wide end oriented towards the front of the subject's torso; and e) a restraint band for securing the subject's hand on the injured side of the subject to the support wedge. In other embodiments, at least one shoulder compression pleat is a plurality of pleats that cover the full shoulder of the subject. In still other embodiments, the support wedge holds the humerus of the subject in a neutral anatomic position.

In some embodiments, the sling comprises an elastic material or fabric. In other embodiments, the elastic material or fabric is selected from the group consisting of cotton spandex, polyester spandex, acetate spandex, and fabric-sided neoprene.

In some embodiments, the pouch further comprises a lining. In other embodiments, the lining comprises an elastic material or fabric. In still other embodiments, the elastic material or fabric is selected from the group consisting of cotton spandex, polyester spandex, acetate spandex, and fabric-sided neoprene.

In some embodiments, fasteners are used to connect parts of the sling system together, such as the first and second ends of the pouch, the arm flap fastener, and the fastener to hold the edge of the wedge flap closed against the inside of the sling. The fasteners may be any means for connecting two elements of the sling system together. In other embodiments, the fasteners are selected from the group consisting of Velcro®, a button, a snap, a hook, and a tie.

In some embodiments, the arm flap stretches around both the support wedge and the arm on the injured side of the subject. In other embodiments, the arm flap stretches around the arm on the injured side of the subject and not around the support wedge. In still other embodiments, the support wedge is secured around the torso of the subject using an adjustable support wedge strap.

In some embodiments, the pouch further comprises a wedge flap that wraps around both the support wedge and the arm on the injured side of the subject. In other embodiments, the pouch further comprises a fold to facilitate the function of the wedge flap. In still other embodiments, the wedge flap further comprises a fastener to hold the edge of the wedge flap closed against the inside of the sling.

In some embodiments, the pouch further comprises at least one other pleat or dart. In other embodiments, the pouch further comprises at least one pocket. In still other embodiments, at least one pocket is capable of holding at least one item selected from the group consisting of an ice pack, a mobile phone, a small purse, and coins.

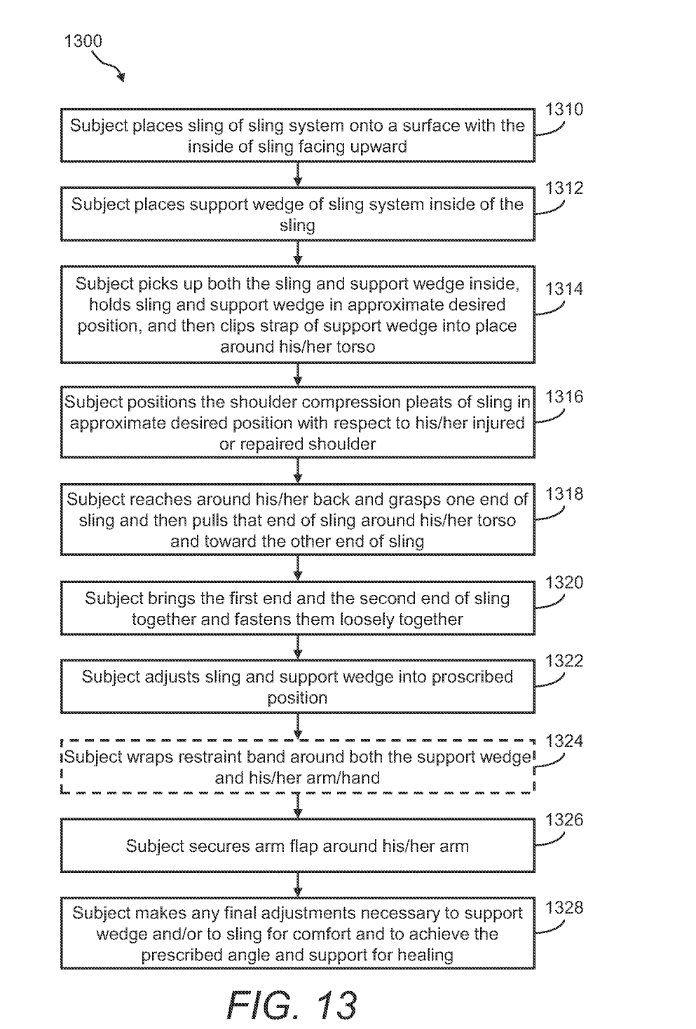

The presently disclosed subject matter also provides methods of using the presently disclosed sling system. FIG. 13 illustrates a flow diagram of an example of a method 1300 of putting on the presently disclosed sling system 100. The method 1300 describes the use of all three components of the sling system 100; namely, method 1300 assumes the use of the sling 110, the support wedge 150, and restraint band 160. The method 1300 includes, but is not limited to, the following steps.

At a step 1310, the subject 170 places the sling 110 of the sling system 100 onto a surface, such as a table or countertop, and then flattens out the sling 110 as much as possible with the inside of the sling 110 facing upward.

At a step 1312, the subject 170 places the support wedge 150 of the sling system 100 into the sling 110 with the narrow end of the support wedge 150 oriented toward the first end 120 of the sling 110 and the wide end of the support wedge 150 oriented toward the second end 122 of the sling 110. In the example of the sporty sling 110, the wedge flap 134 may be wrapped around both the support wedge 150 and the arm of subject 170 and then secured using, for example, Velcro®.

At a step 1314, the subject 170 picks up the sling 110 with the support wedge 150 inside, holds the sling 110 and support wedge 150 in the approximate desired position with respect to his/her torso, and then clips the strap of the support wedge 150 into place around his/her torso.

At a step 1316, using his/her healthy arm and hand (i.e., the arm and hand opposite the injured or repaired shoulder and/or arm), the subject 170 positions the shoulder compression pleats 116 of the sling 110 in the approximate desired position with respect to his/her injured or repaired shoulder or with respect to the shoulder of his/her injured or repaired arm. At this point, the first end 120 of the sling 110 is hanging down loosely onto the back of the subject 170 and the second end 122 of the sling 110 is hanging loosely at the front of the subject 170. The arm flap 118 is also hanging loosely.

At a step 1318, using his/her healthy arm and hand, the subject 170 reaches back around his/her back and grasps the first end 120 of the sling 110 that is hanging down loosely. Then, the subject 170 pulls the first end 120 of the sling 110 around the side of his/her torso and toward the front of his/her torso. That is, the subject 170 pulls the first end 120 of the sling 110 around his/her torso and toward the second end 122 of the sling 110 that is hanging loosely at the front of the subject 170.

At a step 1320, while still holding the first end 120 of the sling 110 in his/her healthy hand, the subject 170 uses his/her other hand (i.e., the hand in the sling 110) to grasp the second end 122 of the sling 110. Then, the subject 170 brings the first end 120 and the second end 122 of the sling 110 together and fastens them loosely together via the fastener 124 at the first end 120 and the fastener 126 at the second end 122. At this point, the sling 110 is fitted loosely around the torso of the subject 170.

At a step 1322, using his/her healthy arm and hand, the subject 170 adjusts the sling 110 and the support wedge 150 into the proscribed position.

At an optional step 1324, the subject 170 rests his/her hand/arm against the support wedge 150 and then uses his/her healthy arm and hand to wrap the restraint band 160 around both the support wedge 150 and his/her arm/hand. The restraint band 160 should be wrapped loosely enough to avoid cutting off blood circulation to the hand, while at the same time be wrapped tightly enough to reliably secure the hand/arm to the support wedge 150, thereby reliably immobilizing the injured or repaired shoulder and/or arm. The restraint band 160 may be particularly useful to the subject 170 when sleeping.

At a step 1326, the subject 170 secures the arm flap 118 around his/her arm. In some embodiments, the arm flap 118 is secured around both the support wedge 150 and the arm of subject 170 (with or without the restraint band 160). In this case, using his/her healthy arm and hand, the subject 170 reaches between his/her torso and the support wedge 150 (or the support wedge strap 152) and grasps the end of the arm flap 118 that is hanging loosely. Then, the subject 170 draws the arm flap 118 up between the support wedge 150 (or the support wedge strap 152) and the his/her torso, then around the support wedge 150 and also around his/her arm and fastens the end of the arm flap 118 to the inside of the sling 110 via the arm flap fastener 128.

In other embodiments, the arm flap 118 is secured around the arm of subject 170, but not around the support wedge 150. In this embodiment, the restraint band 160 is not present. In this case, using his/her healthy arm and hand, the subject 170 reaches between the outside of the support wedge 150 and his/her arm and grasps the end of the arm flap 118 that is hanging loosely. Then, the subject 170 draws the arm flap 118 up around his/her arm and fastens the end of the arm flap 118 to the inside of the sling 110 via the arm flap fastener 128. The arm is now secured within the arm flap 118 and is resting against the support wedge 150.

At a step 1328, the subject 170 makes any final adjustments necessary to the support wedge 150 and/or to the sling 110 for comfort and to achieve the prescribed angle and support for healing.

Accordingly, in some embodiments, the presently disclosed subject matter provides a method for a subject to secure a sling on himself/herself, the method comprising: a) providing a sling system comprising: i) a pouch for receiving a subject's arm and shoulder comprising a shoulder surround portion and an arm surround portion and having a first end and a second end each comprising a fastener; wherein the first and second ends fasten around the torso of the subject at each fastener; and wherein the elbow of the subject is completely covered by the pouch; ii) the arm surround portion further comprising an arm flap comprising an arm flap fastener to stretch around the arm on the injured side of the subject; iii) the shoulder surround portion further comprising at least one shoulder compression pleat; iv) a support wedge comprising a narrow end that is oriented towards the back of the subject's torso and a wide end oriented towards the front of the subject's torso; and v) a restraint band for securing the subject's hand on the injured side of the subject to the support wedge; b) placing the pouch onto a surface with the inside of the pouch facing upward; c) picking up the pouch with the support wedge inside; d) holding the pouch and the support wedge in the approximate desired position to allow securing of the support wedge around the torso of the subject; e) securing the support wedge into place around the torso of the subject; f) positioning the at least one shoulder compression pleat in the approximate desired position with respect to the shoulder; g) reaching around the back of the subject to grasp one end of the pouch and pulling that end of the pouch around the torso toward the other end of the pouch; h) bringing the first end and the second end of the sling together and fastening them loosely together; i) adjusting the pouch and the support wedge into position; and j) securing the arm flap around the arm on the injured side of the subject. In other embodiments, after adjusting the pouch and the support wedge into position, the subject wraps the restraint band around both the support wedge and the arm or hand on the injured side of the subject.

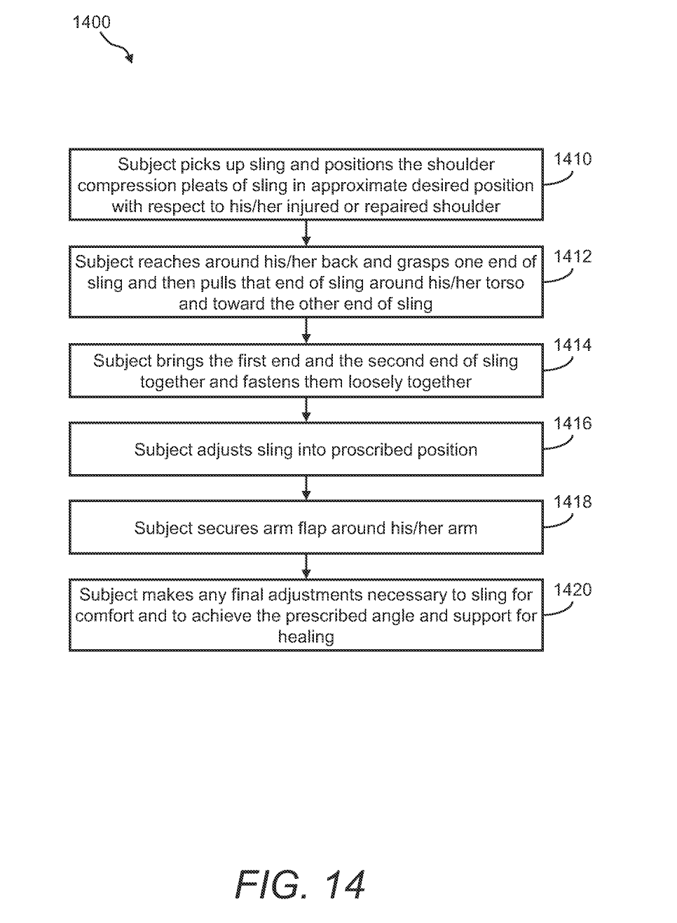

FIG. 14 illustrates a flow diagram of another embodiment of a method 1400 of putting on the presently disclosed sling system 100. The method 1400 describes the use of only the sling 110 of the sling system 100. The method 1400 includes, but is not limited to, the following steps.

At a step 1410, using his/her healthy arm and hand (i.e., the arm and hand opposite the injured or repaired shoulder and/or arm), the subject 170 positions the shoulder compression pleats 116 of the sling 110 in the approximate desired position with respect to his/her injured or repaired shoulder or with respect to the shoulder of his/her injured or repaired arm. At this point, the first end 120 of the sling 110 is hanging down loosely onto the back of the subject 170 and the second end 122 of the sling 110 is hanging loosely at the front of the subject 170. The arm flap 118 is also hanging loosely.

At a step 1412, using his/her healthy arm and hand, the subject 170 reaches back around his/her back and grasps the first end 120 of the sling 110 that is hanging down loosely. Then, the subject 170 pulls the first end 120 of the sling 110 around the side of his/her torso and toward the front of his/her torso. That is, the subject 170 pulls the first end 120 of the sling 110 around his/her torso and toward the second end 122 of the sling 110 that is hanging loosely at the front of the subject 170.

At a step 1414, while still holding the first end 120 of the sling 110 in his/her healthy hand, the subject 170 uses his/her other hand (i.e., the hand in the sling 110) to grasp the second end 122 of the sling 110. Then, the subject 170 brings the first end 120 and the second end 122 of the sling 110 together and fastens them loosely together via the fastener 124 at the first end 120 and the fastener 126 at the second end 122. At this point, the sling 110 is fitted loosely around the torso of the subject 170.

At a step 1416, using his/her healthy arm and hand, the subject 170 adjusts the sling 110 into the proscribed position.

At a step 1418, the subject 170 secures the arm flap 118 around his/her arm. For example, using his/her healthy arm and hand, the subject 170 reaches between his/her torso and his/her arm and grasps the end of the arm flap 118 that is hanging loosely. Then, the subject 170 draws the arm flap 118 up around his/her arm and fastens the end of the arm flap 118 to the inside of the sling 110 via the arm flap fastener 128.

At a step 1420, the subject 170 makes any final adjustments necessary to the sling 110 for comfort and to achieve the prescribed angle and support for healing.

In some embodiments, the presently disclosed subject matter provides a method for a subject to secure a sling on himself/herself, the method comprising: a) providing a sling system comprising: i) a pouch for receiving a subject's arm and shoulder comprising a shoulder surround portion and an arm surround portion and having a first end and a second end each comprising a fastener; wherein the first and second ends fasten around the torso of the subject at each fastener; and wherein the elbow of the subject is completely covered by the pouch; ii) the arm surround portion further comprising an arm flap comprising an arm flap fastener to stretch around the arm on the injured side of the subject; and iii) the shoulder surround portion further comprising at least one shoulder compression pleat; b) picking up the pouch and positioning the at least one shoulder compression pleat in the approximate desired position with respect to the shoulder; c) reaching around the back of the subject to grasp one end of the pouch and pulling that end of the pouch around the torso toward the other end of the pouch; d) bringing the first end and the second end of the sling together and fastening them loosely together; e) adjusting the pouch into position; and f) securing the arm flap around the arm on the injured side of the subject.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Presently Disclosed Sling System and Rotator Cuff Surgery Applications

By way of example, the benefits and advantages of the presently disclosed sling system 100 can be realized with respect to using the sling system 100 following rotator cuff surgery.

With respect to rotator cuff surgery, the repaired rotator cuff tendons take about six weeks to heal initially to the bone. Additionally, it takes the repaired rotator cuff tendons about three months to form a relatively strong attachment to the bone, and about six to nine months before the rotator cuff tendons are completely healed to the bone. According to the publication entitled “A Patient Guide to Failed Rotator Cuff Repairs” by Edward G McFarland MD, Maad Al-Saati MD, Juan Garzon-Muvdi MD, Sonal Sodha, and Gaelle (see http://www.hopkinsortho.org/orthopedicsurgery/FailedRotatorCuff.pdf), the failure rate for rotator cuff surgeries is substantial. The re-tear rate may depend on several major variables, such as the size of the torn tendon, the patient's age, the healing condition of the tendons at the 6-month mark after surgery, and whether the patient has consistently immobilized the repaired tendons post-operatively.

With respect to immobilizing the repaired rotator cuff tendons post-operatively, the currently accepted protocol is for the patient to wear a prescribed shoulder and arm support as well as a support wedge 100% of the time for the first six weeks after rotator cuff surgery. That is, the patient must wear the shoulder and arm support with the support wedge continuously 24 hours per day (even when sleeping) for the first six weeks after rotator cuff surgery. Following the initial 6-week period, the patient must then wear the shoulder and arm support with the support wedge during all of his/her waking hours, but not during his/her sleeping hours. An example of the standard prescribed shoulder and arm support is the Breg® Slingshot brace supplied by Breg, Inc. (Carlsbad, Calif.), which also includes the Breg® Neutral Wedge (see http://www.breg.com/products/shoulder-bracing/slingshot-neutral).

However, there are drawbacks of using the standard prescribed shoulder and arm supports that prompt patients not to wear their shoulder and arm support as prescribed. Therefore, patient compliance with respect to wearing shoulder and arm supports as prescribed is poor and the risk of rotator cuff re-tear and/or of poor healing is high.

As described hereinabove, there are many drawbacks to using the currently prescribed shoulder and arm supports. These drawbacks are disincentives for patients to comply with wearing their shoulder and arm supports as prescribed. Consequently, the risk of rotator cuff re-tear and/or of poor healing is high.

By contrast and referring again to FIG. 1 through FIG. 14, the sling system 100 offers features to substantially mitigate the drawbacks of the standard prescribed shoulder and arm supports for rotator cuff post-operative treatment. The features of the sling system 100 include, but are not limited to, the following:
1) The sling 110 of the sling system 100 is worn on and supported by the injured shoulder or the shoulder of the injured arm, thereby posing no restrictions to the healthy, uninjured shoulder and arm.
2) The sling 110 of the sling system 100 wraps around the entire shoulder and arm as well as around the torso. In so doing, the weight of the arm is redistributed away from the shoulder alone and to the combination of both the shoulder and the torso, thereby relieving the injured shoulder of much of the weight of the arm. Namely, while the shoulder is gently compressed within the confines of the sling 110, the arm is being lifted and carried within the sling 110, wherein the sling 110 is wrapped around the torso in such a way that the majority of the weight of the arm is redistributed away from the shoulder to the torso.
3) The sling 110 of the sling system 100 wraps around the entire shoulder and arm, holding the shoulder and arm in the prescribed position and is easily adjustable to achieve the prescribed tilt or angle for healing.
4) The sling 110 of the sling system 100 includes full shoulder compression pleats that provide a comforting, gentle, and even pressure directly on the injured or repaired shoulder, thereby providing incentive for subjects to wear the sling system 100 as prescribed.
5) The sling 110 of the sling system 100 provides cocoon-like protection of the elbow, thereby reducing, preferably entirely eliminating, the likelihood of the subject's elbow accidentally catching on obstacles and being accidentally bumped or jarred.
6) In the sling system 100, by using the restraint band 160 to secure the arm/hand to the support wedge 150, the wearer's natural reflex to reach for things is mitigated.
7) The sling system 100 is comfortable even when sleeping because it is lightweight and does not include bulky straps and bulky fasteners.
8) In the sling system 100, by using the restraint band 160 to secure the arm/hand to the support wedge 150, the wearer's injured or repaired shoulder and/or arm is held reliably immobilized even when sleeping. Namely, the sling system 100 can be worn comfortably and securely during sleep without fear of the sling 110 slipping off.
9) The sling system 100 can be made aesthetically pleasing to the eye. For example, the sling 110 of the sling system 100 can be implemented as the “dressy” sling 110 or the “sporty” sling 110.
10) The sling 110 of the sling system 100 allows the hand of the injured resting arm to be used for non-lifting, non-pressure types of tasks, such as holding small objects.
11) The sling 110 of the sling system 100 may include other features, such as pockets for holding small items and/or pockets for holding ice packs.

Additionally, in rotator cuff post-operative treatment, there is a certain time period (e.g., about 2 weeks) immediately following surgery in which fluid must be allowed to drain from the surgical site. Therefore, optionally, the sling 110 of the sling system 100 may include an opening 165 in the shoulder surround portion 112 of the sling 110, an example of which is shown in FIG. 6. The opening 165 is sized large enough to allow drainage from the surgical site, while at the same time is sized small enough to not compromise the function of the sling system 100.

The aforementioned features of the sling system 100 provide incentive for subjects to wear the sling system 100 as prescribed, which in turn ensures efficacy of the sling system 100 with respect to immobilizing and healing the injured or repaired shoulder and/or arm.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A sling system with reliable immobilization and comfort for supporting an injured arm or shoulder comprising:
a) a generally rectangular or oval pouch for receiving a subject's arm and shoulder comprising a shoulder surround portion situated lengthwise along the top of the pouch and an arm surround portion situated lengthwise along the bottom of the pouch and having a narrowed first end and a second end each comprising a fastener; wherein the first and second ends fasten around the torso of the subject at each fastener; and wherein the elbow of the subject is completely covered by the pouch;
b) the arm surround portion further comprising an arm flap comprising an arm flap fastener to stretch around the arm on the injured side of the subject;
c) the shoulder surround portion further comprising at least one shoulder compression pleat;
d) a support wedge having a generally elongated block shape and comprising a narrow end that is oriented towards the back of the subject's torso and a wide end oriented towards the front of the subject's torso; and
e) a restraint band for securing the subject's hand on the injured side of the subject to the support wedge.

2. The sling system of claim 1, wherein the at least one shoulder compression pleat is a plurality of pleats that cover the full shoulder of the subject.

3. The sling system of claim 1, wherein the sling comprises an elastic material or fabric.

4. The sling system of claim 3, wherein the elastic material or fabric is selected from the group consisting of cotton spandex, polyester spandex, acetate spandex, and fabric-sided neoprene.

5. The sling system of claim 1, wherein the pouch further comprises a lining.

6. The sling system of claim 5, wherein the lining comprises an elastic material or fabric.

7. The sling system of claim 6, wherein the elastic material or fabric is selected from the group consisting of cotton spandex, polyester spandex, acetate spandex, and fabric-sided neoprene.

8. The sling system of claim 1, wherein any of the fasteners are selected from the group consisting of Velcro®, a button, a snap, a hook, and a tie.

9. The sling system of claim 1, wherein the arm flap stretches around both the support wedge and the arm on the injured side of the subject.

10. The sling system of claim 1, wherein the arm flap stretches around the arm on the injured side of the subject and not around the support wedge.

11. The sling system of claim 1, wherein the support wedge is secured around the torso of the subject using an adjustable support wedge strap.

12. The sling system of claim 1, wherein the pouch further comprises a wedge flap that wraps around both the support wedge and the arm on the injured side of the subject.

13. The sling system of claim 12, wherein the pouch further comprises a fold to facilitate the function of the wedge flap.

14. The sling system of claim 13, wherein the wedge flap further comprises a fastener to hold the edge of the wedge flap closed against the inside of the sling.

15. The sling system of claim 1, wherein the support wedge holds the humerus of the subject in a neutral anatomic position.

16. The sling system of claim 1, wherein the pouch further comprises at least one other pleat or dart.

17. The sling system of claim 1, wherein the pouch further comprises at least one pocket.

18. The sling system of claim 17, wherein the at least one pocket is capable of holding at least one item selected from the group consisting of an ice pack, a mobile phone, a small purse, and coins.

19. A method for a subject to secure a sling on himself/herself, the method comprising:
a) providing a sling system comprising: i) a pouch for receiving a subject's arm and shoulder comprising a shoulder surround portion and an arm surround portion and having a first end and a second end each comprising a fastener; wherein the first and second ends fasten around the torso of the subject at each fastener; and wherein the elbow of the subject is completely covered by the pouch; ii) the arm surround portion further comprising an arm flap comprising an arm flap fastener to stretch around the arm on the injured side of the subject; iii) the shoulder surround portion further comprising at least one shoulder compression pleat; iv) a support wedge comprising a narrow end that is oriented towards the back of the subject's torso and a wide end oriented towards the front of the subject's torso; and v) a restraint band for securing the subject's hand on the injured side of the subject to the support wedge;
b) placing the pouch onto a surface with the inside of the pouch facing upward;
c) picking up the pouch with the support wedge inside;
d) holding the pouch and the support wedge in the approximate desired position to allow securing of the support wedge around the torso of the subject;
e) securing the support wedge into place around the torso of the subject;
f) positioning the at least one shoulder compression pleat in the approximate desired position with respect to the shoulder;
g) reaching around the back of the subject to grasp one end of the pouch and pulling that end of the pouch around the torso toward the other end of the pouch;
h) bringing the first end and the second end of the sling together and fastening them loosely together;
i) adjusting the pouch and the support wedge into position; and
j) securing the arm flap around the arm on the injured side of the subject.

20. The method of claim 19, wherein after adjusting the pouch and the support wedge into position, the subject wraps the restraint band around both the support wedge and the arm or hand on the injured side of the subject.

21. A method for a subject to secure a sling on himself/herself, the method comprising:
a) providing a sling system comprising: i) a pouch for receiving a subject's arm and shoulder comprising a shoulder surround portion and an arm surround portion and having a first end and a second end each comprising a fastener; wherein the first and second ends fasten around the torso of the subject at each fastener; and wherein the elbow of the subject is completely covered by the pouch; ii) the arm surround portion further comprising an arm flap comprising an arm flap fastener to stretch around the arm on the injured side of the subject; and iii) the shoulder surround portion further comprising at least one shoulder compression pleat;
b) picking up the pouch and positioning the at least one shoulder compression pleat in the approximate desired position with respect to the shoulder;
c) reaching around the back of the subject to grasp one end of the pouch and pulling that end of the pouch around the torso toward the other end of the pouch;
d) bringing the first end and the second end of the sling together and fastening them loosely together;
e) adjusting the pouch into position; and
f) securing the arm flap around the arm on the injured side of the subject.
